(19) United States
(12) Patent Application Publication
(10) Pub. No.: US 2005/0239150 A1
(43) Pub. Date: Oct. 27, 2005

(54) Method for diagnosing sepsis by determining anti-asialogangliside antibodies

(76) Inventor: Andreas Bergmann, Berlin (DE)

Correspondence Address: MAYER, BROWN, ROWE & MAW LLP, 1909 K STREET, N.W., WASHINGTON, DC, 20006, US

(21) Appl. No.: 10/516,618

(22) PCT Filed: Apr. 2, 2003
(86) PCT No.: PCT/EP03/03449

§ 371 Date: May 25, 2005

(30) Foreign Application Priority Data
Jun. 4, 2002 (EP) 02012516.7

(51) Int. Cl. G01N 33/53; G01N 33/537; G01N 33/543

(52) U.S. Cl. 435/7.93

(57) ABSTRACT

The invention relates to a method for the early detection and detection, the progression prognosis and the evaluation of the degree of severity, and the treatment-accompanying progression evaluation of sepsis and sepsis-like systemic infections, and for the estimation of the danger which would be presented to patients at high risk of sepsis, by the development of a sepsis. According to the inventive method, the presence and/or quantity of anti-asialo-G<SB>M1</SB> antibodies (anti-AG<SB>M1</SB> antibodies), and antibodies which cross-react with the same, in a biological liquid of a patient or a patient at high risk of sepsis is determined, and conclusions are drawn from the presence and/or quantity of the same in terms of presence, expected progression, degree of severity or the success of a treatment for the inflammatory disease or sepsis, or in terms of the danger presented to a patient at high risk of sepsis. The inventive method enables potentially harmful banked blood to be eliminated if donor blood is used.

The present invention relates to a novel method for the medical diagnosis, in particular the preventive diagnosis, of sepsis and sepsis-like systemic inflammatory diseases and, derived therefrom, also a method for monitoring donor blood, for example in the context of screening of blood banks. It is based on the detection for the first time of greatly increased concentrations of anti-ganglioside autoantibodies, in particular of anti-asialo-G_{M1} autoantibodies and antibodies cross-reacting therewith, for example anti-GM₁ antibodies, of the IgG and IgA type, in the sera of patients suffering from sepsis.

In particular, the present invention relates to a method for the early diagnosis of a septic reaction in a human patient (patient at risk of sepsis), in which, owing, for example, to a preceding medical intervention and/or a trauma (accident, burn, war injuries, decubitus and the like), there is an increased risk of the development of a septic reaction, and for the estimation of the potential risk to a patient from a septic reaction before, for example, a medical intervention or immediately after a trauma, if these are of a type such that a sepsis can develop as a dangerous complication thereafter.

The method is particularly valuable as a risk exclusion method, i.e. for eliminating an acute danger from sepsis as a result of a negative test for the above-mentioned antibodies.

Inflammations are defined very generally as certain physiological reactions of an organism to different types of external effects, such as, for example, injuries, burns, allergens, infections by microorganisms, such as bacteria, fungi and viruses, to foreign tissues which trigger rejection reactions, or to certain endogenous states of the body which trigger inflammation, for example in autoimmune diseases and cancer. Inflammations may occur as harmless, localized reactions of the body but are also typical features of numerous serious chronic and acute diseases of individual tissues, organs, organ parts and tissue parts.

Local inflammations are generally part of the healthy immune response of the body to harmful effects, and hence part of the life-preserving defence mechanism of the organism. However, if inflammations are part of a misdirected response of the body to certain endogenous processes, such as, for example, in autoimmune diseases, and/or are of a chronic nature, or if they reach systemic extents, as in the case of systemic inflammatory response syndrome (SIRS) or in a severe sepsis caused by infection, the physiological processes typical of inflammatory reactions go out of control and become the actual, frequently life-threatening pathological process.

It is now known that the origin and the course of inflammatory processes are controlled by a considerable number of substances which are predominantly of a protein or peptide nature or are accompanied by the occurrence of certain biomolecules for a more or less limited time. The endogenous substances involved in inflammatory reactions include in particular those which can be assigned to the cytokines, mediators, vasoactive substances, acute phase proteins and/or hormonal regulators. The inflammatory reaction is a complex physiological reaction in which both endogenous substances activating the inflammatory process (e.g. TNF-α, interleukin-1) and deactivating substances (e.g. interleukin-10) are involved.

In systemic inflammations, as in the case of sepsis or of septic shock, the inflammation-specific reaction cascades spread in an uncontrolled manner over the whole body and become life-threatening in the context of an excessive immune response. Regarding the knowledge appearing in the relevant published literature and relating to the occurrence and the possible role of individual groups of endogenous inflammation-specific substances, reference is made, for example, to A. Beishuizen et al., “Endogenous Mediators in Sepsis and Septic Shock”, Advances in Clinical Chemistry, Vol. 33, 1999, 55-131; and C. Gabay et al., “Acute Phase Proteins and Other Systemic Responses to Inflammation”, The New England Journal of Medicine, Vol. 340, No. 6, 1999, 448-454. Since the understanding of sepsis and related systemic inflammatory diseases, and hence also the recognized definitions, have changed in recent years, reference is also made to K. Reinhart et al., “Sepsis und septischer Schock” [Sepsis and Septic Shock], in: Intensivmedizin, Georg Thieme Verlag, Stuttgart, New York, 2001, 756-760, where a modern definition of sepsis is given. In the context of the present Application, the terms sepsis and inflammatory diseases used are based on the definitions as given in the three stated references.

Whereas at least in Europe the systemic bacterial infection detectable by a positive blood culture long characterized the term sepsis, sepsis is now primarily understood as being systemic inflammation which is caused by infection but, as a pathological process, has considerable similarities with systemic inflammations which are triggered by other causes. Said transformation in the understanding of sepsis has resulted in changes in the diagnostic approaches. Thus, the direct detection of bacterial pathogens was replaced or supplemented by complex monitoring of physiological parameters and, more recently, in particular by the detection of certain endogenous substances involved in the sepsis process or in the inflammatory process, i.e. specific “biomarkers”.

An introduced endogenous substance which is particularly suitable as a sepsis biomarker is procalcitonin. Procalcitonin is a prohormone whose serum concentrations reach very high values under the conditions of a systemic inflammation of infectious aetiology (sepsis), whereas it is virtually undetectable in healthy persons. High values of procalcitonin are also reached in a relatively early stage of a sepsis so that the determination of procalcitonin is also suitable for early diagnosis of a sepsis or for early distinguishing of a sepsis caused by infection from severe inflammations which have other causes. The determination of procalcitonin as a sepsis marker is the subject of the publication by M. Assicot et al., “High serum procalcitonin concentrations in patients with sepsis and infection”, The Lancet, Vol. 341, No. 8844, 1993, 515-518; and the patents DE 42 27 454 C2 and EP 0 656 121 B1 and U.S. Pat. No. 5,639,617. In recent years, the number of publications on the subject of procalcitonin has greatly increased. Reference may therefore also be made to W. Karzai et al., “Procalcitonin—A New Indicator of the Systemic Response to Severe Infection”, Infection, Vol. 25, 1997, 329-334; and M. Oczenski et al., “Procalcitonin: a new parameter for the diagnosis of bacterial infection in the peri-operative period”, European Journal of Anaesthesiology 1998, 15, 202-209; and furthermore H. Red1 et al., “Procalcitonin release patterns in a baboon model of trauma and sepsis: Relationship to cytokines and neopterin”, Crit Care Med 2000, Vol. 28, No. 11, 3659-3663; and H. Red1 et al., “Non-Human Primate Models of Sepsis”, in: Sepsis 1998; 2:243-253; and the further literature references cited therein, as typical recent published reviews.

The availability of the sepsis marker procalcitonin has given considerable impetus to sepsis research, and intensive efforts are now being made to find further biomarkers which can supplement the procalcitonin determination and/or are capable of providing additional information for purposes of fine diagnosis or differential diagnosis. Results of these efforts are to be found in numerous patent applications of the Applicant, in particular in DE 198 47 690 A1 or WO 00/22439, and in a number of still unpublished German (DE 101 19 804.3 or PCT/EP02/04219; DE 101 31 922.3; DE 101 30 985.6) or European Patent Applications (EP 01128848.7; EP 01128849.5; EP 01128850.3; EP 01128851.1; EP 01128852.9; EP 01129121.8; EP 02008840.7 and EP 02008841.5). Reference is hereby made to the content of said patents and patent applications for supplementing the present description.

Since the endogenous substances formed during inflammations are part of the complex reaction cascade of the body, not only are such substances also of diagnostic interest but attempts are also currently being made, with considerable effort, to intervene therapeutically in the inflammatory process by influencing the origin and/or the concentration of individual substances of this type, in order to stop at as early a stage as possible the systemic spread of the inflammation which is observed, for example, in sepsis. In this context, endogenous substances which can be shown to be involved in the inflammatory process are also to be regarded as potential therapeutic targets. Attempts starting from certain mediators of the inflammatory process to influence this therapeutically in a positive manner are described, for example, in E. A. Panacek, “Anti-TNF strategies”, Journal für Anästhesie und Intensivbehandlung; No. 2, 2001, 4-5; T. Calandra et al., “Protection from septic shock by neutralization of macrophage migration inhibitory factor”, Nature Medicine, Vol. 6, No. 2, 2000, 164-170; or K. Garber, “Protein C may be sepsis solution”, Nature Biotechnology, Vol. 18, 2000, 917-918. In view of the fairly disappointing results of such therapeutic approaches to date, there is considerable interest in identifying further endogenous biomolecules which are as inflammation- or sepsis-specific as possible and, as therapeutic targets, also open up new prospects for success for fighting inflammation.

All above-mentioned biomarkers or biomarkers mentioned in said prior applications are physiological peptides or protein molecules which have, for example, enzyme character or the character of (pro)hormones or are defined cell fragments. Proteins of the immunoglobulin type, in particular of the IgG and IgA type, i.e. antibodies, have not been discussed to date as diagnostically relevant biomarkers for sepsis, in particular a sepsis caused by bacteria, or for a particular risk situation with regard to the genesis of sepsis or in a progressing sepsis.

It is a basic object of the present invention to provide a further sepsis parameter which can be determined in the context of sepsis diagnosis and sepsis prevention and, if appropriate, permits the initiation of measures for sepsis prophylaxis and sepsis prevention.

This object is achieved by a method according to claim 1 and the preferred embodiments thereof according to claims 2 to 9.

Further methods for health care are derived from the method of claims 1 to 9 and may be designated as methods for monitoring donor blood and exogenous substances and are summarized in claims 10 and 12 and in claims 11 and 13 relating back to these.

Further embodiments of the present invention are evident from the following explanations and examples.

The present invention is based in general on the extremely surprising finding that, where possible to check experimentally at the time, a certain antibody or autoantibody known per se in other contexts is found diagnostically at significantly increased levels with extraordinary frequency in tested sera of patients suffering from sepsis, whereas the same antibody is not detectable or detectable only in substantially smaller amounts in healthy normal persons. The serum samples of patients suffering from sepsis, in which the antibody was found at significantly increased levels, had been taken from patients for a considerable part only shortly after an event giving rise to the risk of sepsis (for example about 2 h after, for example, an operation, an accident, a burn), which patients only subsequently develop the full symptoms of a sepsis. The occurrence of specific antibodies with high sensitivity (correct detection of samples of patients suffering from sepsis) at such an early time in the development of sepsis was very surprising and has important consequences with regard to the genesis of such antibodies and the negative role which they play or may play in the context of a sepsis process. This will be discussed in more detail below.

By means of the determination, according to the invention, of anti-asialo-G_{M1} antibodies and ganglioside antibodies cross-reacting therewith, i.e. binding to asialo-G_{M1}, it is possible, according to the present invention, to detect with high reliability a susceptibility to the development of sepsis in the context of an increased individual risk situation or an acute risk due to an already developing sepsis in the case of patients at risk of sepsis. At the same time, in the event of a negative result of the antibody determination, those patients for whom such an increased risk is not present or in whom no development of sepsis has begun are identified.

If the determined antibody also occurs in increased concentrations in some other specific diseases, a correct interpretation of the results of measurements is as a rule possible without great difficulties for the purposes of sepsis diagnosis taking into account additional clinical findings and history of a patient at risk of sepsis. This applies both to the known neuropathies, in which anti-ganglioside antibodies are found, and for cancer patients in whom such antibodies are likewise usually increased. The increase in anti-ganglioside antibodies in the sera of cancer patients is a subject of the slightly prior, unpublished Patent Application EP 02009884.4 of the Applicant, in which it is shown that the same antibodies which are determined in the sepsis diagnosis method according to the invention can also serve as universal biomarkers for malignant neoplastic diseases of virtually any type. Reference is hereby made to the entire content of said prior Application and the parallel Patent Application EP 02009882.8 relating to therapeutic aspects, in particular regarding the general explanations of gangliosides and of the previously known role of anti-ganglioside antibodies, for supplementing the statements in the present Application.

As will be explained further below, there is furthermore reason to assume that anti-asialo-G_{M1} antibodies found at a significantly increased level in sepsis—as also in malignant neoplasms, cf. EP 02009884.4 and EP 02009882.8—and antibodies cross-reacting therewith play a possibly decisive disease-promoting role in the course of both sepsis and the origin of cancer as a result of adversely influencing the normal immune response, in particular in the context of destruction or inhibition/deactivation of the cytotoxic lymphocytes which are referred to as “natural killer cells” (NK cells) and are an important member of the immune response.

It is therefore important not to unintentionally supply such antibodies to a patient from the outside, for example with the administration of donor blood, and furthermore to determine those factors from the human environment which may lead to sensitization with respect to the formation of such anti-asialo antibodies in normal persons. This gives rise to further aspects of the present invention which are explained below, namely firstly the determination of such antibodies for monitoring banked blood or in donor blood for the purpose of screening of banked blood (blood bank screening) and, secondly, the testing of substance-bound environmental factors for their potential ability to give rise to the formation of these antibodies by molecular mimicry.

In further aspects, the present invention therefore also relates to methods for the determination of anti-asialo-G_{M1} antibodies and antibodies cross-reacting therewith, in which these are not determined in patient sera for sepsis or cancer diagnosis, but are determined for monitoring donor blood, for example in banked blood, in order to avoid damaging the immune response of a patient to whom this blood is administered, in particular by deactivating his NK cells and destroying their function. Such a determination does not differ fundamentally from a determination of the same antibodies in a blood sample (serum sample) of a patient, and only the origin of the blood sample and the purpose of carrying out the determination (screening) are different.

As also explained briefly below, it is already known that the formation of antibodies reactive with gangliosides, such as G_{M1}, can also be triggered in a human by bacterial exposure (e.g. infections with Campylobacter jejuni or Heliocobacter pylori) (cf. the corresponding statements in the prior Application EP 02009884.4 and the literature mentioned therein). It is therefore to be assumed that there are further molecular structures which resemble the carbohydrate moiety of gangliosides and can therefore potentially give rise to the formation of anti-AG_{M1} antibodies or antibodies cross-reacting therewith in a similar manner to said bacteria by molecular mimicry in humans, and can occur in the human environment in a very wide range of forms. It is therefore a further object, which is derived from the diagnostic results described in this Application and in said prior applications of the Applicant, to provide a method which makes it possible to identify in the environment substances which simulate gangliosides, in particular asialo-G_{M1}, as such and thus to determine an otherwise possibly undetectable risk of such substances for human health.

This purpose can be served by a screening method in which substances suitable as such substances are brought into contact with an assay system comprising anti-AG_{M1} antibodies and specific binders therefor, e.g. AG_{M1} in immobilized form, and the influence of the substances to be tested manifesting itself as competition, on the binding of the anti-AG_{M1} antibodies to their specific binders used in the assay is determined. In such a test, for example, sera of patients for whom high antibody titres were measured can be used directly as a source for anti-AG_{M1} antibodies, and the relative competitive impairment of the specific binding to the specific binder can be determined in substantially the same manner as in the determination of the antibodies in a serum or in a blood sample, except that a foreign substance to be tested is also added to the reaction mixture and the result obtained is compared with a reference value for the substance-free reaction mixture. From this point of view, the present invention therefore also relates to a method which can be regarded as a method for environmental screening.

Below, the discovery of the method according to the invention for sepsis diagnosis, with presentation of the measured values on which this method is based, and a currently preferred procedure for carrying it out in practice will be explained in more details.

An interpretation and ex-post plausibility check of the method according to the invention in the light of scientific publications which can be related to the subject matter of the present invention will then be described, and these show that the anti-ganglioside antibodies or autoantibodies determined according to the invention, in particular anti-AG_{M1} antibodies and antibodies cross-reacting therewith, play a decisive role for the sepsis process, in particular for the genesis of a sepsis and the course thereof, owing to their influence on the function of the NK cells, which on the one hand provide information about new therapeutic approaches for sepsis prevention, sepsis inhibition and, if appropriate, sepsis therapy and from which the above-mentioned further preventive aspects of the present invention can be derived.

The present invention is a result of the intensive researches by the Applicant in the area of clinical diagnosis of autoimmune diseases and of sepsis. In the present case, the research is started from the knowledge that certain anti-ganglioside antibodies also belong to the antibodies which are discussed in the literature in association with autoimmune diseases, in particular nerve-damaging, neuropathic autoimmune diseases.

Gangliosides are glycolipids which are constituents of the extracellular side of the plasma membrane of animal cells and as such also occur in nerve tissue. They contain several monosaccharide units per mole but have no phosphorus content and are assigned to the sphingolipids. Compared with proteins, they tend to be low molecular weight biomolecules. The gangliosides to which the antibodies discussed in the context of the present invention bind are primarily the asialo-G_{M1} abbreviated to AG_{M1} in the present Application and the associated monosialo-ganglioside which is abbreviated to G_{M1} and for which the Applicant was able to show that the antibody populations found in sera or at least predominant parts are bound selectively by both gangliosides (AG_{M1} and/or G_{M1}) G_{M1} is a ganglioside which has a polysaccharide chain of 4 sugar monomer units which comprise two D-galactose units, one N-acetylgalactosamine unit and one D-glucose unit, the latter being bound to a so-called ceramide moiety. In the ganglioside G_{M1}, an N-acetylneuraminic acid radical (NANA; sialic acid or o-sialinic acid radical; “monosialo” radical), which is missing in the sialinic acid-free asialo-G_{M1} (AG_{M1}), is bound to the D-galactose unit arranged inside the polysaccharide chain.

Said gangliosides and related compounds are associated with numerous important biological functions of the human body, including, for example, axonal growth and neuronal differentiation, receptor functions and participations in various immune reactions of the body and in signal transduction and cell-cell recognition. Further details are to be found, for example, in the publications mentioned in the list of references for the prior Application EP 02009884.2 already mentioned.

It has long been known that antibodies or autoantibodies which bind to said and related gangliosides can occur in the human body. The physiological role of such antibodies and their possible importance for clinical diagnosis are the subject of numerous scientific investigations.

By far the predominant part of all published papers are concerned with the role and the diagnostic significance of anti-ganglioside antibodies in neuropathies, for example in immunomediated motor neuropathies, such as Guillain-Barre syndrome (radiculoneuritis, polyradiculitis) and the related (Miller-)Fisher syndrome. An increased occurrence of anti-G_{M1} autoantibodies in some patients was also reported in association with Alzheimer's disease. Furthermore, they were also found in individual HIV patients. Attempts to determine them in association with certain types of cancer had provided contradictory results which were not very informative or results with low sensitivity, before the Applicant carried out its own investigations, the results of which are recorded in the stated unpublished prior Patent Applications EP 02009884.4 and EP 02009882.1.

If the relevant scientific publications on the subject of the “determination of anti-ganglioside antibodies” are studied in more detail, it is evident that the findings and information regarding the amounts to be observed—which as a rule tend to be low—and types of different (auto)antibodies in the various patients and pathological states differ to a relatively great extent from one another, with great similarity of many observations. This was sufficient to lead to the conclusion that the determination of such antibodies is only of limited to doubtful value for clinical diagnosis (cf. for example Michael Weller et al., Ganglioside antibodies: a lack of diagnostic specificity and clinical utility? J Neurol (1992) 239:455-459).

However, the Applicant presumed that the reasons for the literature data, which diverge considerably in some cases, could lie in the methodology and that, owing to systematic errors of the measuring methods used, it might not have been possible to date to obtain any truly reliable, informative and consistent results. Most of the determinations for which the results were published relate to patients having neurological disturbances and were carried out by means of immunoassays of the ELISA type, which were designed so as to employ a solid phase to which gangliosides—in some cases obtained by the authors themselves from biological material—were bound. This solid phase was reacted with the liquid biological sample in which the antibodies to be determined were presumed to be present. After the incubation time chosen in each case, a solid-liquid separation and washing of the solid phase, human antibodies bound to said phase were then marked unspecifically with enzyme-marked animal anti-human Ig antibodies and determined.

When applied to the determination of anti-ganglioside antibodies, an assay of said type is extremely susceptible to disturbances and errors of measurement and can give reliable, reproducible results only on careful standardization and normalization. One of the causes of this is that the quality of the solid phase which is obtained by immobilizing relatively low molecular weight gangliosides is susceptible to strong variations. This is due in part to the fact that remaining free binding capacities of the solid phase have to be saturated prior to the reaction with the liquid sample. As a rule, bovine serum albumin, i.e. a protein, is used for this purpose. However, this step results in the unspecific binding of other proteins, for example those of the IgG type, from the sample becoming very high, which leads to a strong background signal, against which the antibodies to be determined have to be determined. If, however, the sensitivity of an assay is not very high—which as a rule is the case in assays of the ELISA type—background signal and measured signal may be so strongly superposed that incorrect (false negative or false positive) or unreliably reproducible measured results are obtained.

Regarding the various assay methods used and the systematic and practical problems in the application of such methods to the determination of anti-ganglioside antibodies, reference may be made, for example, to: Einar Bech et al., ELISA-Type Titertray Assay of IgM Anti-GM1 Autoantibodies, Clin. Chem. 40/7, 1331-1334 (1994); Alan Pestronk, MD et al., Multifocal motor neuropathy: Serum IgM anti-GM1 ganglioside antibodies in most patients detected using covalent linkage of GM1 to ELISA plates, Neurology 1997, 49:1289-1292; Mepur H. Ravindranath et al., Factors affecting the fine specificity and sensitivity of serum antiganglioside antibodies in ELISA, J. Immunol. Methods 169 (1994) 257-272; Armin Alaedini et al., Detection of anti-GM1 Ganglioside Antibodies in Patients with Neuropathy by a Novel Latex Agglutination Assay, J. Immunoassay, 21(4), 377-386 (2000); Armin Alaedini et al., Ganglioside Agglutination Immunoassay for Rapid Detection of Autoantibodies in Immune-Mediated Neuropathy, J. Clin. Lab. Anal. 15:96-99, 2001. In particular, Mepur H. Ravindranath et al. describe in detail, in said publication, some of the basic problems of the practical anti-ganglioside antibody determination.

In view of this initial situation, the Applicant decided to tackle the problem of the reproducible determination of anti-G_{M1} or anti-AG_{M1} antibodies and their diagnostic significance, for example in Alzheimer patients and to make use of its particular experience and materials as a producer of assays for the clinical diagnosis of autoantibodies. For internal research, the Applicant developed variants of an improved modification of the previously known antiganglioside assays, while maintaining all customary quality standards. The measurements of antibodies binding to Gm or AG_{M1} in sera of a comparative group of normal persons (blood donors) without relevant clinical pathological symptoms and in sera of various persons affected by disease were carried out by means of these improved assays and, as described in the prior Applications EP 02009884.4 and EP 02009882.8, gave, firstly, the surprising result that high significantly increased titres for anti-G_{M1} or anti-AG_{M1} antibodies of the IgA and of the IgG type, but not of the IgM type, were found in all sera available to the Applicant and obtained from patients suffering from cancer, in comparison with normal persons. Secondly, these measurements gave the no less surprising result that a comparable situation also applies to the presence of the corresponding antibodies in sera of patients suffering from sepsis. The present Application is based on these last-mentioned findings and describes technical teachings which are derived therefrom for sepsis prevention and sepsis therapy and health care generally.

Although the results described in more detail below were obtained using a certain improved ligand binding assay (“immunoassay”) from the Applicant's laboratory, the use of the knowledge obtained is possible not only with an assay of the special format described. Rather, it is assumed that the specific assay described below is even substantially suboptimal for the relevant antibody determination, and that commercial assays for the clinical determination of anti-ganglioside antibodies, in particular of anti-AG_{M1} and anti-G_{M1} (auto)antibodies will differ substantially in several respects from the described assay after optimization.

The methods for the determination of said antibodies in a biological sample may be any known immunodiagnostic methods which are used for the selective detection and for the measurement of the amounts of antibodies (autoantibodies). Preferably, the antibodies are determined with the aid of a ligand binding assay in which the respective ganglioside in immobilized form is used as an antigen for binding the antibodies sought. For marking the antibodies specifically bound from a biological sample, anti-human antibodies marked in some suitable manner known per se, marked ganglioside derivatives or binders simulating the carbohydrate structure thereof and having an affinity suitable for the respective assay format can then be used.

Competitive assay formats may also have particular advantages. Preferably, instead of employing enzyme marking, another marker is chosen, for example a marker for a chemiluminescence detection reaction, e.g. an acridinium ester. It is of course preferable to use for the antibody determination an assay which ensures the required high sensitivity in the range of the antibody concentrations occurring and permits separation of the measured signals from the assay background.

The assay method can furthermore be adapted to chip technology or designed as an accelerated test (point-of-care test), it also being possible to carry out the antibody determination according to the invention as part of a multiparameter determination in which at least one further sepsis parameter or infection parameter is simultaneously determined and in which a measured signal in the form of a set of at least two measured quantities is obtained and is evaluated more exactly for the fine diagnosis of sepsis or infection. Further parameters of this type are to be regarded as being those which are selected from the group consisting of the parameters which in some cases are known or are disclosed in the above-mentioned prior patent applications of the Applicant, i.e. from the group consisting in particular of procalcitonin, CA 125, CA 19-9, S100B, S100A proteins, soluble cytokeratin fragments, in particular CYFRA 21, TPS and/or soluble cytokeratin-1 fragments (sCY1F), the peptides inflammin and CHP, peptide prohormones, glycine N-acyltransferase (GNAT), carbamoylphosphate synthetase 1 (CPS 1) and fragments thereof and the C-reactive protein (CRP) or fragments of all proteins mentioned.

It may be advantageous to carry out the multiparameter determination as a simultaneous determination by means of a chip technology measuring apparatus or an immunochromatographic measuring apparatus, in which the evaluation of the complex measured results obtained by means of the measuring apparatus is carried out with the aid of a computer program.

In order to avoid unjustifiably narrow and restrictive interpretations of the terms used in the present Application and the associated claims, some of the most important terms are to be defined in particular below for the purposes of the present Application:

“Antibody”: This term includes, without distinguishing between different methods of genesis and formation, antibodies both against external antigens and against endogenous structures, i.e. autoantibodies, where the latter may also have become autoantibodies by antigen cross-reactions from antibodies against external antigens and may have preserved their binding capability with respect to external antigens.

When, for example, it is stated that an antibody binds “to ganglioside structures and to antigen structures simulating ganglioside structures” or is “reactive towards gangliosides or certain gangliosides”, where reactive means “reactive in the context of specific binding”, it should be sufficiently defined by this definition without, for example, its specific binding also to additional other antigen structures, or its practical determination using reagents (for immobilization or marking or as competitors) with molecular structures which only simulate AG_{M1}, in particular the carbohydrate structure thereof, playing a role for the definition as antibodies according to the invention.

“Cross-reacting” When it is stated that antibodies cross-reacting with anti-asialo-G_{M1} antibodies also are to be/can be determined, this means primarily antibodies which bind in the context of a cross reaction in a comparable manner to asialo-G_{M1} structures as are to be found as determinants on NK cells, and may therefore have physiological effects comparable to those of anti-asialo-G_{M1} antibodies with regard to these NK cells.

“Ganglioside” In the context of the present invention, the term “ganglioside” primarily represents the gangliosides AG_{M1} in the characterization of the binding behaviour of the antibodies to be determined. However, the term is also intended to include related gangliosides not investigated to date, e.g. fucosylated gangliosides, if it is found that antibodies binding to these gangliosides and having a comparable diagnostic significance are found in sepsis sera.

“Assay” This term covers any highly sensitive ligand binding assays suitable for a determination of the (auto)antibodies in question, without a restriction to a certain assay format (sandwich assay, competitive assay, agglutination assay) or a certain type of marking being desired. Of course, certain assay formats and/or markers are superior to others and are therefore preferred (for example chemiluminescence marking compared with enzyme marking). The use of an assay which is worse or better than the assay described specifically below is, however, not intended to lead out of the scope of the claims if it serves for diagnostic purposes defined in the present Application.

“Sensitivity” In the context of the present invention, a high sensitivity means that the antibodies are found in at least 50%, better 70%, preferably at least 85% and even more preferably at least 95% of all patients suffering from sepsis.

Further meanings of terms are evident to a person skilled in the art from the introductory and following description of the invention and its embodiments.

In the description below, reference is made to figures which show the following:

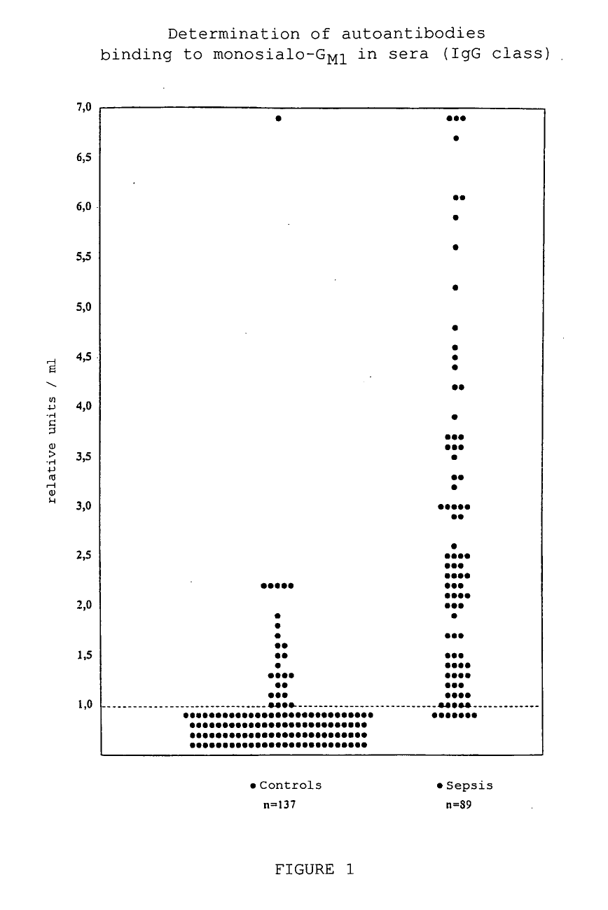

FIG. 1 shows a graph of the results of the measurement of antibodies of the IgG class which bind to monosialo-G_{M1}, in sera of 137 control persons, compared with the results of the measurement of 89 sera of patients suffering from sepsis;

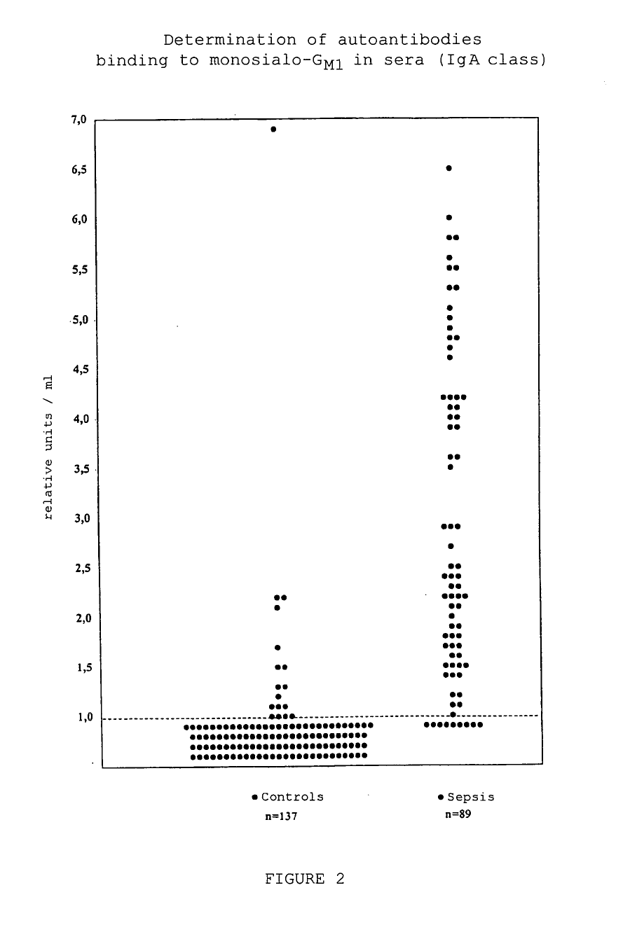

FIG. 2 shows the results of a measurement of the same sera as in FIG. 1 for antibodies of the IgA class which bind to monosialo-G_{M1};

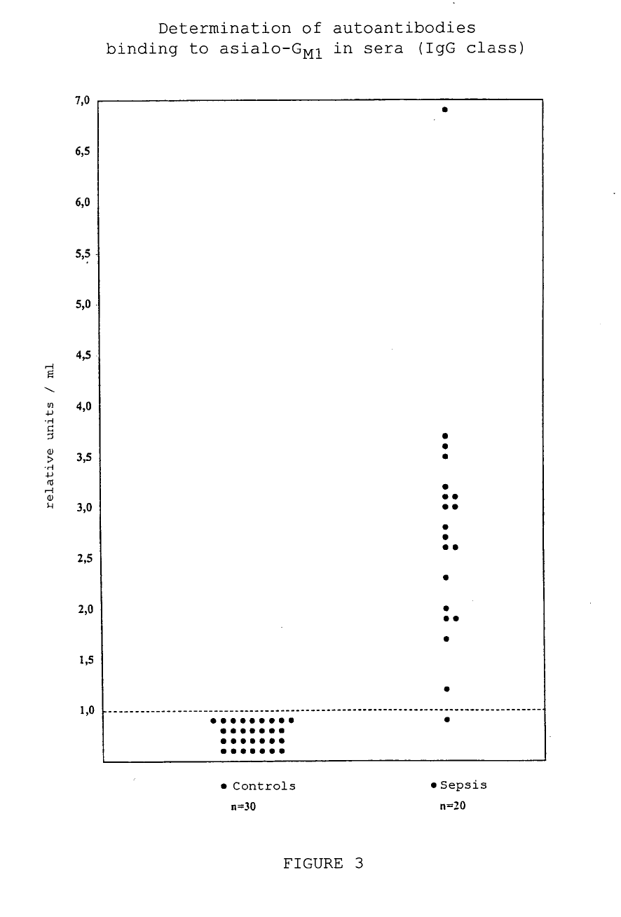

FIG. 3 shows the results of the determination of antibodies of the IgG class which bind to asialo-G_{M1}, in sera of 30 normal persons (controls), compared with the results of the measurement of 20 sera of patients suffering from sepsis (all sera are partial groups of the sera measured in FIGS. 1 and 2);

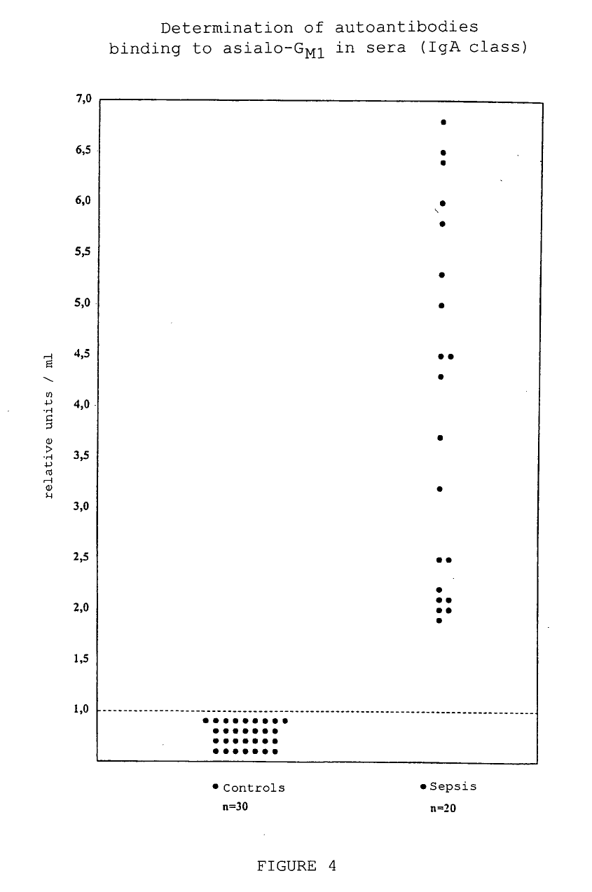

FIG. 4 shows the results of the determination of antibodies of the IgA class which bind to asialo-G_{M1}, in the same sera as in FIG. 3.

### ANTIBODY ASSAYS

1. Preparation of the Assay Components:

A. Preparation of Test Tubes (Coated Tubes; CT)

Three types of test tubes were prepared: (a) test tubes to which the gangliosides G_{M1} and AG_{M1} were bound, and (b) test tubes having a BSA coating for the determination of the background signal specific to the sample.

a) For the preparation of the ganglioside-coated test tubes (GA-CTs), the gangliosides (G_{M1} and AG_{M1}, in each case obtained from Sigma, Germany) were dissolved in methanol and then diluted in PBS (phosphate-buffered saline solution), pH 7.2, 25% methanol, to a concentration of 5 μg/ml. In each case 300 μl of this solution were introduced into polystyrene tubes (“Star” polystyrene tubes from Greiner, Germany) and incubated at room temperature for 16 h. Thereafter, the content of the tubes was removed by means of suction, and the tubes were filled with 4.5 ml of 0.5% BSA (bovine serum albumin, protease-free, from Sigma, Germany) in water for saturating free binding sites and incubated for 2 h at room temperature. Thereafter, the tube content was decanted, and the tubes were filled with 0.2% Tween, 10 mM Tris/HCl, 10 mM NaCl, pH 7.5, and decanted again. The tubes were then used for the antibody assay.

(b) Since serum constituents bind to the BSA used for saturating free binding sites of the test tube wall, and the degree of such binding may be very different in the case of different sera, it is necessary to determine a background signal specific to the sample separately for each serum.

For this purpose, the same test tubes were filled with 4.5 ml of 0.5% BSA in water and incubated for 2 h at room temperature. Thereafter, the tube content was decanted, and the tubes were filled with 0.2% Tween, 10 mM Tris/HCl, 10 mM NaCl, pH 7.5, and decanted again. The tubes (HR-CTs) were then used for the determination of the background signal specific to the sample.

B. Preparation of Acridinium Ester-Marked Anti-Human IgG and Anti-Human IgA Antibodies (Tracers)

Goat anti-human IgG antibodies (affinity-purified; grade II, from Scantibodies, USA) and goat anti-human IgA antibodies (affinity-purified; from Sigma, Germany), in each case 2 mg/ml in PBS, pH 7.4, 100 μl, were each mixed with 10 μl of acridinium NHS ester (from Hoechst, Germany, 1 mg/ml in acetonitrile; cf. DE 36 28 573 A1) and incubated for 20 min at room temperature. After addition of 300 μl of 20 mM glycine, 50 mM NaCl, the marked antibodies were purified by means of adsorption chromatography via hydroxyapatite HPLC. The separation column used was an HPHT column (120 mm×8 mm), equilibrated in solvent A (1 mM NaPO₄, pH 7.0, 10% methanol, 0.1% Lubrol; “LM A”; Lubrol 17A17 was obtained from Serva, Germany). The flow rate was 0.8 ml/min. Bound antibodies were eluted by means of a linear 40 min gradient of LM A/LM B (500 mM NaPO₄, pH 7.0, 10% methanol, 0.1% Lubrol; “LM B”) at a flow rate of 0.8 ml/min. The column outflow was measured continuously for UV absorption at 280 nm (protein) and 368 nm (acridinium ester). Acridinium esters not bound to protein were eluted in unbound form from the column and thus completely separated from the marked antibodies. The antibodies were eluted in about 25 min. After the determination of the protein concentration (BCA method) of the HPLC-purified marked antibodies, the tracers were diluted to a final concentration of 0.1 μg/ml in PBS, pH 7.2, 1 mg/ml of goat IgG (from Sigma, Germany) and 1% BSA.

2. Carrying Out the Determination of Anti-Ganglioside Antibodies

The samples to be investigated (human sera) were diluted 1:20 with PBS, pH 7.2, 1 mg/ml of goat IgG, 1% BSA. In each case 10 μl thereof were pipetted into GA-CTs or HR-CTs. Incubation for 16 hours with shaking (IKA mechanical shaker KS250 basic, 400 rpm) at 4° C. was then effected.

Unbound antibodies were removed by filling/decanting of the tubes 5 times with 1 ml of 0.2% Tween, 10 mM Tris/HCl, 10 mM NaCl, pH 7.5. Antibodies remaining on the tube surfaces were detected by binding of marked goat anti-human IgG or marked goat anti-human IgA, by incubating the tubes with, in each case, 200 μl of the respective tracer (cf. above, 1.B.) and then for 3 h at 4° C. with shaking. Unbound tracer was removed by washing 5 times (as above).

The amount of the marked antibody which remained on the tube surface was measured by means of luminescence measurement in a Berthold LB.952T/16 luminometer.

The luminescence signal of each sample, obtained for GA-CTs, was corrected by the respective background signal for the same sample, measured with the HR-CTs. The resulting signal (differential signal) is the signal for antibodies binding to the gangliosides G_{M1} or AG_{M1} and originating from the respective sample. Dilution series of samples having a high content of anti-ganglioside antibodies were used as relative calibrators for the quantification.

3. Measurement of Sera of Healthy Normal Persons (Controls) and Patients Suffering from Sepsis

The following series measurements were carried out using the test tubes prepared as described and using the method described above:

Control Sera:

137 control sera (blood donor sera and—for avoiding age-related influences on the antibody concentrations—sera of normal persons of various ages from old people's homes and the Applicant's employees) were used as control sera for the antibody determinations using GA-CTs which were coated with G_{M1}. For the antibody determinations using GA-CTs which were coated with AG_{M1}, a partial group of these sera which comprised only 30 sera was measured.

Test Sera:

89 sera of patients suffering from sepsis were used as test sera for the antibody determinations using GA-CTs which were coated with G_{M1}. For the antibody determinations using GA-CTs which were coated with AG_{M1}, 20 sera of patients suffering from sepsis (partial group of the above-mentioned 89 sera) were used. For each test serum, there existed clinical documentation which related, inter alia, to the patient's history, the time of sampling and the subsequent course of sepsis.

The results of the determinations of antibodies of the classes IgG and IgA using GA-CTs which were coated with G_{M1} are shown in FIGS. 1 and 2.

The results of the determinations of antibodies of the classes IgG and IgA using GA-CTs which were coated with AG_{M1} are shown in FIGS. 3 and 4.

4. Discussion of the Results of the Determination of Anti-Ganglioside Antibodies in Control Sera and in Sera of Patients Suffering from Sepsis

As impressively shown by the measured results summarized in FIGS. 1 to 4, the determination of antibodies of the classes IgA and/or IgG which bind to gangliosides (AG_{M1} and/or G_{M1}) permits a clear distinction of the control group from the patients suffering from sepsis, by virtue of the fact that substantially increased AG_{M1} and G_{M1} antibody titres are found in virtually all (82 out of 89, i.e. 92%) of the sepsis sera investigated. It appears that the determinations of IgA using AG_{M1}-coated test tubes give measured results with the highest sensitivity (all patients suffering from sepsis are positive) and selectivity (no detection of non-sepsis patients as positive) (although a restriction to be taken into account is that, for practical reasons, the number of only 20 determinations was smaller than in the case of the 89 determinations using G_{M1}-coated tubes).

It is furthermore important that, in an experiment also to determine corresponding antibodies of the IgM type analogously to the determinations of antibodies of the IgG and IgA type, no levels for antibodies of the IgM type which were increased to a diagnostically relevant extent were found in the sepsis sera (results not shown).

The detection of substantially increased concentrations of antibodies of the IgA and IgG type also in patient's serum samples which had been obtained only a short time (about 2 h) after the “sepsis risk event” (e.g. operation, accident, burn), and the lack of evidence of antibodies of the IgM type, ruled out the possibility that the detected antibodies were formed only as a result of the “sepsis risk event” or of a bacterial infection associated therewith. However, this means that either the antibodies are already present beforehand in the respective patients suffering from sepsis and/or that the activation of the presensitized immune system of the patient in the manner of a “booster” effect, triggered by the sepsis risk event, has initiated intensive antibody production.

The fact that the AG_{M1} or G_{M1} antibodies were found at substantially increased levels in substantially all measured sepsis sera (92%) is therefore to be interpreted so as to mean that the formation of a sepsis is either causally linked to the prior presence of the antibodies in question in the respective patient or is at least the consequence of activation of “molecular machinery” (in the form of B-cells) already present in the patient owing to prior immunization, which begins its intensive antibody production under the influence of the “sepsis risk event” or of an infection associated therewith. Patients without these antibodies or the presensitization required for their rapid production probably do not develop a sepsis or develop one only with difficulty, based on the experimental results to date.

The observed results can be made plausible: it is known that the so-called “natural killer cells” (NK-cells; cytotoxically active lymphocytes) have, on their surface, asialo-G_{M1} structures to which anti-AG_{M1} antibodies can specifically bind and thus deactivate the NK-cells. Reference may therefore be made to the fact that it is usual in the field of animal experiments which employs experimental animals in which tumours are to be artificially produced to eliminate the immune defence of the experimental animal by administering anti-AG_{M1} antibodies in combination with a carcinogen or a tumour nucleus, so that the experimental cancer—desired in the animal model—can develop (Hugh F. Pross et al., Role of Natural Killer Cells in Cancer, Nat Immun 1993; 12:279-292; Lewis L. Lanier et al., Arousal and inhibition of human NK Cells, Immunological Reviews 1997, Vol. 155:145-154; Yoichi Fuki et al., IgG Antibodies to AsialoGM1 Are More Sensitive than IgM Antibodies to Kill in vivo natural Killer Cells and Prematured Cytotoxic T Lymphocytes of Mouse Spleen, Microbiol. Immunol. Vol. 34(6), 533-542, 1990; N. Saijo et al., Analysis of Metastatic Spread and Growth of Tumor Cells in Mice with Depressed Natural Killer Activity by Anti-asialo GM1 Antibody or Anticancer Agents, J Cancer Res Clin Oncol (1984) 107: 157-163; Sonoku HABU et al., Role of Natural Killer Cells against Tumor growth in Nude Mice—A Brief Review, Tokai J Exp Clin Med., Vol. 8, No. 5, 6: 465-468, 1983; Lewis L. Lanier, NK Cell Receptors, Annu. Rev. Immunol. 1998, 16: 359-93; Theresa L. Whiteside et al., The role of natural killer cells in immune surveillance of cancer; Current Opinion in Immunology 1995, 7:704-710; Tuomo Timonen et al, Natural Killer cell-target cell interactions, Current Opinion in Cell Biology 1997, 9:667-673).

However, active NK-cells play an extremely important role in the human immune defence, also in the case of sepsis or severe bacterial infections. Thus, for example, Shuiui Seki et al., in: Role of Liver NK Cells and Peritoneal Macrophages in Gamma Interferon and Interleukin-10 Production in Experimental Bacterial Peritonitis in Mice, Infection and Immunity, Vol. 66, No. 11, 1998, 5286-5294, describe the important role of NK-cells for the production of inflammation-promoting and anti-inflammatory cytokines. They show that switching off the NK cells artificially with experimental use of anti-AG_{M1} antibodies leads to inhibition of the production of the anti-inflammatory interferon-γ. Effects of surgical stress and an endotoxin-induced sepsis on the NK-cell activity has also already been described, in particular in: P. Toft et al., in: The effect of surgical stress and endotoxin-induced sepsis on the NK-cell activity, distribution and pulmonary clearance of YAC-1 and melanoma cells, APMIS 1999; 107:359-364. A possible influence of physiologically formed antibodies with NK-cell reactivity is not taken into account in any of the papers mentioned.

The detection of naturally occurring anti-AG_{M1} antibodies and anti-ganglioside antibodies cross-reacting therewith, e.g. anti-G_{M1} antibodies, and the increased levels of such antibodies in sera of patients suffering from sepsis mean, however, that such antibodies represent a previously unconsidered parameter influencing the infection- or inflammation-specific cytokine cascade, in that they intervene in the natural cytokine regulation cycle and, by disturbing or switching off the NK-cells, can cause this to malfunction and trigger a septic reaction in the patient.

Since, as already explained, owing to their nature and the time of their occurrence, the anti-AG_{M1} antibodies found in sepsis sera cannot have formed as a result of the acute sepsis-inducing event or a new infection associated therewith, but must already have been present at least as a predisposition, the determination of such antibodies is also suitable for determining the risk situation and for the prognosis in the case of a patient who is to be classed as a sepsis risk patient.

The measured high sensitivity therefore makes the determination of anti-AG_{M1} antibodies and anti-ganglioside antibodies cross-reacting therewith, in particular of those of the IgG and/or IgA classes, a promising assay method for sepsis diagnosis, in particular for early diagnosis and, as explained above, also for the determination of the personal risk situation of a sepsis risk patient or the prognosis of a sepsis.

Discoveries which might suggest the method according to the invention cannot be found in the scientific literature. There were only a few known papers in which anti-ganglioside antibodies were determined in association with severe acute infectious diseases. Such a case is Chagas disease, caused by the parasite Trypanosoma cruzi (cf. D. H. Bronia et al., in: Ganglioside treatment of acute Trypanosoma cruzi infection in mice promotes long-term survival and parasitological cure, Annals of Tropical Medicine & Parasitology, Vol. 93, No. 4, 341-350 (1999) and the literature cited therein). The last-mentioned paper speculates that an observed, substantially advantageous effect of administration of exogenous gangliosides to mice infected with the parasite T. cruzi might induce in said mice the production of anti-ganglioside antibodies which react with the glycolipids of the membrane of T. cruzi and thus cause the death of the parasite. On the basis of the findings in the present Application, such an explanation is not very probable: owing to their NK cell-inhibiting effect, the anti-ganglioside antibodies observed in Chagas disease are not a healing-promoting but a disease-inducing or disease-promoting factor. By administering exogenous, scarcely antigenic gangliosides, the anti-AG_{M1} antibodies are in fact not formed but probably blocked, with the result that the effect of the NK-cells is restored in the mice (or in patients) and the immune system of the parasite can dominate. The known paper cannot indicate any relationship at all between anti-asialo-G_{M1} antibodies and the origin and worsening of a sepsis and can therefore neither anticipate nor suggest the method according to the invention.

The interpretation of the findings which are the basis of the present invention can be extended as follows: the sensitization of a patient with regard to the production of anti-ganglioside antibodies in reaction to an antigenic stimulation may have been brought about by a general infection or optionally also corresponding environmental substances and thereafter remain latent for a long time. However, once the production of anti-asialo-G_{M1} antibodies has been initiated or greatly increased in a human individual, for example owing to bacterial exposure (e.g. infections with Campylobacter jejuni or Heliobacter pylori), this patient fulfils the preconditions that, in certain physiological stress situations with high NK-cell activity (for example cell degeneration due to mutagenic events; a sepsis risk situation), the NK-cells and hence the immune defence will be damaged, resulting in an increased risk that a defence reaction which is triggered by, for example, a “sepsis stress” and requires intervention by the NK-cells would also stimulate the production of the above-mentioned antibodies, and that these will then cancel out the effect of the NK-cells. The control cycle of the immune response is then decisively disturbed, and a sepsis may develop.

It should therefore rather be assumed that anti-AG_{M1} antibody titres found at significantly increased levels in all sepsis sera which were measured in the above-mentioned determinations are one of the preconditions for the origin of the sepsis, and the presence of such antibodies has a negative effect.

Since the antibodies cross-reacting with gangliosides and the effect on the immune system which is necessary for the production thereof must already be present before the development of a sepsis, the determination of anti-AG_{M1} antibodies can advantageously be carried out according to the invention also in the context of the determination of a disposition, i.e. as a determination of a sepsis risk marker. In this context, it may be advantageous to carry out such a determination after an in vivo stimulation of the antibody formation of a sepsis risk patient, for example before an operation, using safe stimulants. In view of the IgA antibodies found at substantially increased levels (cf. FIGS. 2 and 4), the antibody determination should also be capable of being carried out by suitable assays in body secretions (e.g. saliva, mucous).

The above statements also show the importance of avoiding external supply of anti-AG_{M1} antibodies, for example with donor blood, to a patient, in particular in a situation where the functioning of his immune system has to meet high requirements, and as far as possible avoiding exposure of persons generally with regard to antigenic substances which simulate ganglioside structures and can thus lead to the formation of anti-ganglioside antibodies or antibodies cross-reacting with ganglioside structures.

The consequences which result from the findings described in this Application and relate to novel methods for the prevention, inhibition and therapy of septic pathological conditions and for general health care form the subject matter of a separate parallel patent application filed simultaneously with the present Application.

1. Method for the early diagnosis and diagnosis, for the prognosis and the assessment of the severity and for the therapy-accompanying assessment of the course of sepsis and sepsis-like systemic infections and for the estimation of the risk of a sepsis risk patient through the formation of a sepsis, characterized in that the presence and/or amount of anti-asialo-G_{M1} antibodies (anti-AG_{M1} antibodies) and antibodies cross-reacting therewith in a biological fluid of a patient or sepsis risk patient are determined and conclusions are drawn from the presence and/or amount thereof with regard to the presence, the expected course, the severity or the success of a therapy of the inflammatory disease or sepsis or with regard to the risk of a sepsis risk patient.

2. Method according to claim 1, characterized in that anti-AG_{M1} and/or anti-G_{M1} (auto)antibodies of the IgG and/or IgA type are determined.

3. Method according to claim 1, characterized in that the biological fluid is blood, a blood fraction or a secretion.

4. Method according to claim 1, characterized in that the determination is carried out with the aid of a ligand binding assay of the sandwich type or of the competitive type or of an agglutination assay.

5. Method according to claim 1, characterized in that the determination of the antibodies in a blood sample of a sepsis risk patient is carried out after prior in vivo and/or in vitro stimulation of the antibody production.

6. Method according to claim 1, characterized in that it is carried out as part of a multiparameter determination, in which at least one further inflammation or infection parameter is simultaneously determined and in which a measured result in the form of a set of at least two measured parameters is obtained, which result is evaluated for the fine diagnosis of sepsis.

7. Method according to claim 6, characterized in that, in addition to the anti-ganglioside autoantibodies, at least one further parameter which is selected from the group consisting of the proteins procalcitonin, CA 125, CA 19-9, S100B, S100A proteins, LASP-1, soluble cytokeratin fragments, in particular CYFRA 21, TPS and/or soluble cytokeratin-1 fragments (sCY1F), the peptides inflammin and CHP, peptide prohormones, glycine N-acyltransferase (GNAT), carbamoylphosphate synthetase 1 (CPS 1) and the C-reactive protein (CRP) or fragments thereof is determined as part of the multiparameter determination.

8. Method according to claim 6, characterized in that the multiparameter determination is carried out as a simultaneous determination by means of a chip technology measuring apparatus or of an immunochromatographic measuring apparatus.

9. Method according to claim 8, characterized in that the evaluation of the complex measured result obtained using the measuring apparatus is carried out with the aid of a computer program.

10. Method for the quality control of donor blood for medical purposes, in which the presence and/or amount of anti-asialo-G_{M1} antibodies (anti-AG_{M1} antibodies) and antibodies cross-reacting therewith, in particular anti-G_{M1} antibodies, are determined in a sample of the donor blood and, in the case of positive detection of such antibodies,
the donor blood is rejected or
is subjected to an affinity purification for removing the antibodies determined and is administered to a patient only after a subsequent further antibody determination with a negative result.

11. Method according to claim 10, in which the donor blood investigated is banked blood from a blood bank or freshly obtained donor blood.

12. Method for discovering and for detecting individual substances or constituents of mixtures of substances, which have structural properties which simulate ganglioside structures, in which individual substances or mixtures of substances to be investigated are tested in an assay system which is based on the binding of anti-ganglioside antibodies to a specific binder and the detection of bound antibodies, a competitive reduction of the antibody binding to the specific binder in the presence of the substance to be investigated being regarded as an indication of
antibody-blocking properties of the substance or
a potential risk of the substance owing to an antigen effect with initiation of the production of anti-AG_{M1} antibody or antibodies cross-reacting therewith in humans.

13. Method according to claim 12, in which the individual substances or mixtures of substances which are used for human or animal nutrition and/or are administered to humans for medical or cosmetic reasons are tested.
